(19) United States
(12) Patent Application Publication
(10) Pub. No.: US 2026/0076718 A1
(43) Pub. Date: Mar. 19, 2026

(54) BONE FIXATION DEVICE WITH SECURED REMOTE OPERATION

(71) Applicant: Synthes GmbH, Oberdorf (CH)

(72) Inventors: Oren Cohen, Moreshet (IL); Shahar Harari, Tel-Aviv (IL)

(73) Assignee: Synthes GmbH, Oberdorf (CH)

(21) Appl. No.: 18/885,278

(22) Filed: Sep. 13, 2024

(51) Int. Cl. A61B 17/66; A61B 17/00; G16H 40/67

(52) U.S. Cl. CPC A61B 17/66; G16H 40/67; A61B 2017/00212

(57) ABSTRACT

Reconfiguration of a bone fixation device of a patient may include generating, on a main server, a new actuation plan for the bone fixation device, the new actuation plan including operational parameters of the bone fixation device, notifying a patient device that the new actuation plan is available on the main server, requesting from a patient on the patient device an approval of the new actuation plan. Upon receiving the patient's approval, requesting by the patient device, from the main server, the new actuation plan from the main server, and reconfiguring the bone fixation device with the new actuation plan.

### TECHNICAL FIELD

The present disclosure thereof, relates to medical devices, and, more particularly, it concerns secure and authorized remote operation of an adjustable bone fixation device.

### BACKGROUND

U.S. Pat. No. 11,600,368 to Austin and Mason discloses methods and devices for implementation of a health care prescription, where some devices may automatically trigger a second prescription via the patient device. Managing bone fixation devices, especially those operated by patients in non-clinical settings, often requires periodic adjustments to ensure that the treatment remains effective and responsive to the patient's evolving needs. Changes in the patient's condition, such as variations in healing speed, increased pain, or other unforeseen clinical developments, may necessitate updates to the device's operational parameters.

Traditionally, adjusting these parameters would involve a visit to a healthcare facility, where clinicians could manually reconfigure the device. This approach, while effective, can be inconvenient for the patient, leading to potential delays in treatment adjustments, and imposing unnecessary strain on both the patient and healthcare resources.

### SUMMARY

The present disclosure addresses the need for a more flexible, patient-centered approach by enabling the remote reconfiguration of a bone fixation device. The system is designed to allow healthcare providers to create and update actuation plans on a central server. Once a new actuation plan is generated, the patient is notified through their patient device, ensuring they are kept informed about the progress of their treatment. Upon approval from the patient, the plan is securely transmitted to the patient's device and applied to the bone fixation device, ensuring that the updates are both timely and secure.

The system may include a requirement for patient approval before the new plan is implemented. This step ensures that patients retain control over their treatment while maintaining the integrity and security of the process. After the patient approves the new plan, it is downloaded to their device and applied to the bone fixation device, eliminating the need for a physical visit to the clinic.

This approach not only enhances patient convenience by allowing for seamless adjustments in their home environment but also ensures that any changes are made securely, and with the patient's informed consent. The method supports timely and personalized adjustments to treatment, ultimately contributing to better patient outcomes and a more efficient use of healthcare resources.

The following is a non-exclusive list of some exemplary embodiments of the disclosure. The present disclosure also includes embodiments which include fewer than all the features in an example and embodiments using features from multiple examples, even if not listed below.

Unless otherwise defined, all technical and/or scientific terms used within this document have meaning as commonly understood by one of ordinary skill in the art/s to which the present disclosure pertains. Regarding exemplary embodiments described below, the materials, methods, and examples are illustrative and are not intended to be necessarily limiting.

Some embodiments of the present disclosure are embodied as a system, method, or computer program product. For example, some embodiments of the present disclosure may take the form of an entirely hardware embodiment, an entirely software embodiment (including firmware, resident software, micro code, etc.) or an embodiment combining software and hardware aspects that may all generally be referred to herein as a “circuit,” “module” and/or “system.”

Implementation of the method and/or system of some embodiments of the present disclosure can involve performing and/or completing selected tasks manually, automatically, or a combination thereof. According to actual instrumentation and/or equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computational device e.g., using any suitable operating system.

In some embodiments, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage e.g., for storing instructions and/or data. Optionally, a network connection is provided as well. User interface/s e.g., display/s and/or user input device/s are optionally provided.

Some embodiments of the present disclosure may be described below with reference to flowchart illustrations and/or block diagrams. For example, illustrating exemplary methods and/or apparatus (systems) and/or computer program products according to embodiments of the present disclosure. It will be understood that each step of the flowchart illustrations and/or block of the block diagrams, and/or combinations of steps in the flowchart illustrations and/or blocks in the block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart steps and/or block diagram block or blocks.

These computer program instructions may also be stored in a computer readable medium that can direct a computer (e.g. in a memory, local and/or hosted at the cloud), other programmable data processing apparatus, or other devices to function in a particular manner, such that the instructions stored in the computer readable medium can be used to produce an article of manufacture including instructions which implement the function/act specified in the flowchart and/or block diagram block or blocks.

The computer program instructions may also be run by one or more computational device to cause a series of operational steps to be performed e.g., on the computational device, other programmable apparatus, and/or other devices to produce a computer implemented process such that the instructions which execute provide processes for implementing the functions/acts specified in the flowchart and/or block diagram block or blocks.

Some of the methods described herein are generally designed only for use by a computer, and may not be feasible and/or practical for performing purely manually, by a human expert. A human expert who wanted to manually perform similar tasks, might be expected to use different methods, e.g., making use of expert knowledge and/or the pattern recognition capabilities of the human brain, potentially more efficient than manually going through the steps of the methods described herein.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates an adjustable bone fixation device connected to a bone, according to embodiments of the presently disclosed subject matter;

FIG. 2 illustrates a control system for use with an adjustable bone fixation device, according to embodiments of the presently disclosed subject matter;

FIG. 3 is an example of an actuation plan for adjustment of a plurality of struts of the bone fixation device, according to embodiments of the presently disclosed subject matter;

FIG. 4 illustrates an exemplary system configuration for remote reconfiguration of a bone fixation device of a patient, according to embodiments of the presently disclosed subject matter;

FIG. 5A illustrates a method and flow of communications with the exemplary system for remote reconfiguration of a bone fixation device of a patient, according to embodiments of the presently disclosed subject matter;

FIG. 5B illustrates a method and flow of communications with the exemplary system for remote reconfiguration of a bone fixation device of a patient, according to embodiments of the presently disclosed subject matter;

FIG. 6 illustrates an exemplary system configuration for remote reconfiguration of a bone fixation device of a patient, according to embodiments of the presently disclosed subject matter;

FIG. 7 illustrates a method and flow of communications with the exemplary system for remote reconfiguration of a bone fixation device of a patient, according to embodiments of the presently disclosed subject matter.

FIG. 8 illustrates a high-level block diagram of the exemplary system for remote reconfiguration of a bone fixation device of a patient, according to embodiments of the presently disclosed subject matter.

### DETAILED DESCRIPTION OF EMBODIMENTS

The present disclosure, in some embodiments, thereof, relates to performing of authorized and secure configuration and re-configuration of a medical device, and more particularly, but not exclusively, to updating a treatment plan (prescription) of an adjustable bone fixation device.

A broad aspect of some embodiments of the disclosure relates to performing remote reconfiguration of a bone fixation device of a patient. The bone fixation device including a frame connectible to bone tissue, a plurality of adjustable struts connected to the frame, and a control system including: a plurality of actuators associated with the plurality of adjustable struts, each actuator configured for adjusting a length of at least one adjustable strut. The method and system may include generating, on a main server, a new actuation plan for the bone fixation device, the new actuation plan including operational parameters of the bone fixation device. Notifying a patient device that the new actuation plan is available on the main server. Requesting from a patient on the patient device an approval of the new actuation plan. Upon receiving the patient's approval, requesting by the patient device, from the main server, the new actuation plan from the main server. Reconfiguring the bone fixation device with the new actuation plan.

The present disclosure is useful when a medical device is operated remotely (for example, not in a clinic/hospital, for example in a patient's home) according to a pre-defined plan and there is a need to update the current plan due to changes in the expected treatment (such as slower or faster implementation) or other clinical need (such as pain). An example of such a medical device is an external fixation system MAXFRAME AUTOSTRUT™ Multi-Axial Correction System (available from DePuy Synthes of Johnson & Johnson, West Chester, Pennsylvania, USA). In such cases, there may be a desire to update the plan remotely, without the need for the patient to come to a clinic/hospital. Requirements for updating may include performing the update with authorization of the patient and securely (only authorized changes).

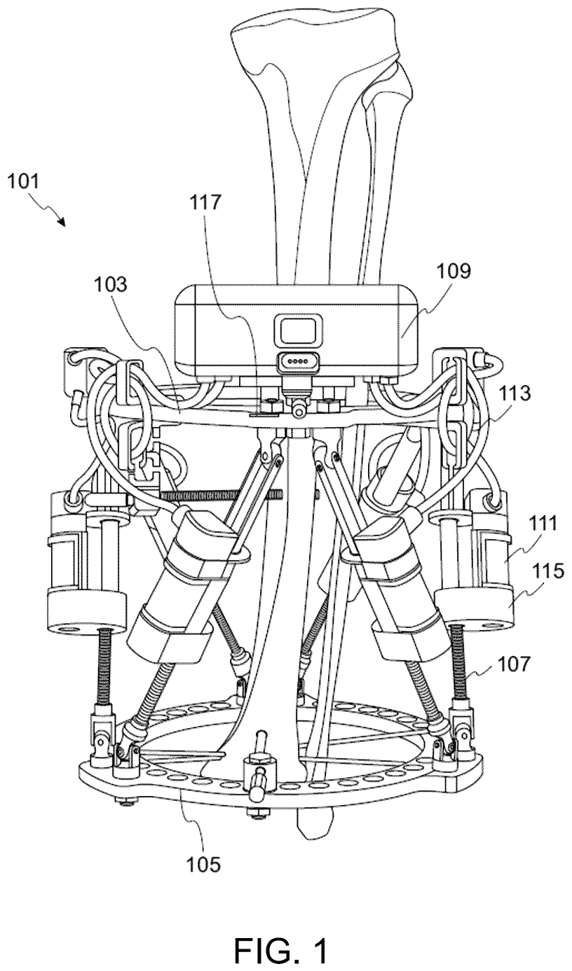

FIG. 1 illustrates an adjustable bone fixation device (or “bone fixation device”) connected to a bone, according to embodiments of the presently disclosed subject matter.

Bone fixation device 101 is generally intended to be connected to the bone of a patient, in a surgical process. In some cases, the device is used for the treatment of a fractured bone, misaligned bone(s), a deformed bone, a bone that needs to be changed in length, and/or other orthopedic or generally bone related conditions.

The bone fixation device generally includes a frame constituting of at least two portions, (first fixation element 103 and a second fixation element 105), and a plurality of struts 107 (e.g., 1, 2, 4, 5, 8, 10 or intermediate or larger number of struts), connecting the at least two portions of the frame. In an exemplary embodiment, 6 struts 107 connect the two portions (103, 105) of the frame, the 6 struts providing, potentially, the ability to control 3D spatial relationship between two frame portions. In some embodiments, as shown, the bone fixation device is shaped as a hexapod, and the two frame portions are formed as two rings having six struts which interconnect the rings. In other embodiments, the two frame portions may include open rings, arc shaped frames, horseshoe shaped frames, rods, and/or otherwise shaped frame portions.

In some embodiments, the two portions of the frame are at least partially connected to the bone via pins (e.g., transfixation pins), rods, wires (e.g., k-wires), or other suitable fixation elements which extend from the frame portion and into the bone. Adjustment of the struts, such as by lengthening or shortening a strut along a linear axis, modifies the distance between the two frame portions (e.g., by pulling on the frame portions towards each other or by pushing the frame portions away from each other. In an example, shortening of struts can approximate the two frame portions towards each other; lengthening of struts can distance the two frame portions away from each other. Adjustment of the struts can also modify the relative position and/or orientation of the two frame portions (and the bone portions to which the frame portions are attached) with respect to each other, for example, shortening of some of the struts and/or lengthening of some of the struts can change an angular orientation of the frame portions with respect to each other (for example, when the frame portions consist of rings, change the plane in which the ring lies and/or change a rotational positioning of the ring).

Adjustment of the bone fixation device is carried out, in accordance with some embodiments, by a control system which is operably connected to the device. The control system generally includes a control unit 109 and a plurality of actuators 111, such as motors (e.g., linear motors). In some embodiments, each of the actuators is associated with a single strut and is configured to drive the adjustment of the specific strut. In some embodiments, the control unit is electrically connected to the actuators via cables 113. (Additionally, or alternatively, a wireless connection may be established between the control unit and the actuators).

In some embodiments, each of the actuators is maintained within a designated adaptor 115 which holds the actuator in an operable coupling with the strut. In some embodiments, the adaptor is shaped to maintain the actuator axially aligned with the strut which the actuator adjusts. Alternatively, in some embodiments, an actuator may be contained (e.g., embedded) within the strut itself.

In some embodiments, the control system of the bone fixation device includes one or more sensors, for example: sensors configured for obtaining system related measurements, such as for measuring operational parameters of the actuators (e.g. torque generated by an actuator, current consumption, operation voltage, rotation speed of the actuator, etc.); and/or sensors configured for obtaining measurements related to the surroundings, such as for measuring environmental conditions (e.g. temperature, humidity), measuring a load or impact on the device, measuring a posture of the patient, etc. FIG. 1 shows an example of a sensor 117 for measuring load acting on the device, positioned, for example, at an attachment area of a strut to the top frame portion.

Note that while a hexapod external fixation device is shown herein, other bone fixation devices such as a monorail are also contemplated.

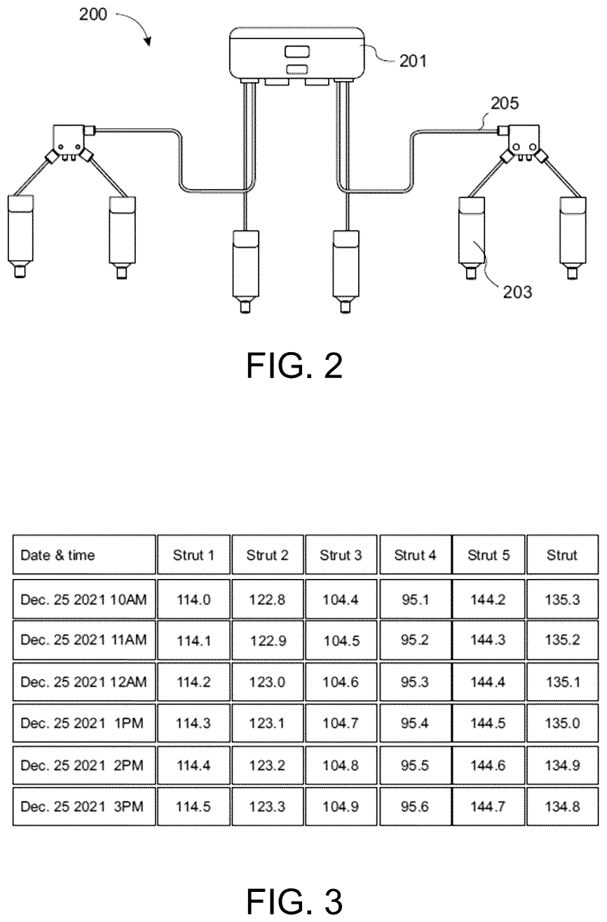

FIG. 2 illustrates a control system for use with an adjustable bone fixation device, according to embodiments of the presently disclosed subject matter.

A control system 200 as shown generally includes a control unit 201, including a housing in which computational, processing, communication and/or memory means are contained. The control unit is operably connected to a plurality of actuators 203, optionally, via cables 205 or other suitable wiring/electric connections. In some embodiments, each of the actuators includes a motor, for example, a brush DC motor or a brushless DC motor.

In some embodiments, control unit 201 includes a power source (not shown), for example an electric power source. In some embodiments, the power source includes a battery, for example a non-replaceable battery or a replaceable battery or a rechargeable battery. In some embodiments, the control unit delivers electric power from the power source to each of the actuators via the cables. Optionally, the battery is sufficient to power the device for as long as the bone fixation device is required to stay connected to the bone, for example, for a time period of between 1-3 months.

In some embodiments, in use, the control system is coupled to the frame and the struts of the bone fixation device. In some embodiments, each actuator is operably connected to a strut such that activation of the actuator generates torque for adjusting the strut. In an example, the actuator rotates a gear or a gear train which is operably coupled to at its end to a threaded lead screw. Movement of the lead screw linearly shortens or lengthens the strut by extending or contracting an adjustable segment of the strut. It is noted that other mechanisms may be used for adjusting the strut, for example, a hydraulic mechanism, a spring-based mechanism, a magnetic mechanism, and/or other mechanism suitable for extending or contracting an adjustable segment of the strut.

In embodiments in which the actuator is external to the strut, the actuator may be held by an adaptor or other suitable restraining means for coupling the actuator to the struts. In embodiments in which the actuator is embedded within the strut, a connector may be used for connecting the control unit (such as via the cables or other wiring) to the embedded actuator.

In some embodiments, the control unit housing is removably connected to the frame, for example via fasteners.

In some embodiments, an actuation plan is uploaded (or otherwise communicated) to the control unit, in which the actuation plan can be stored (for example, in a memory of the control unit). Optionally, an actuation plan is communicated to the control unit over the network.

The actuation plan is designed to carry out a treatment regimen, which can be determined based on a diagnosis of the patient (for example, using the results of tissue imaging); based on patient parameters (e.g., age, level of physical activity); based on the required bone modification; and/or other factors.

In some embodiments, the actuation plan sets parameters according to which the control system operates the actuators. The parameters can be set for each actuator separately, for multiple actuators together, and/or for the bone fixation device in general. The parameters can be set per a single actuation session, or per multiple actuation sessions together. The parameters may also include timing changes and adjustments, such as halting operations, resuming operations, and similar. In one non-limiting example, an actuation plan may include halting operation (only a halt). In another example, an actuation plan may include resuming (only resuming) a current actuation plan. In another example, resuming may be with a new actuation plan. Thus, approval by the patient of the actuation plan may be required for any actuation plan modifications, including the ones that involve only a pause, only a halt, only a resume, or other specific function. Various functions can be implemented using embodiments of the current system and method, and these functions are managed as part of the actuation plan.

Operational parameters set by the actuation plan can include for one or more actuation, for example:
a. Timing of actuation (e.g., an actuation schedule, actuation start time, actuation end time, time intervals (or permitted ranges) between actuation sessions, specific times (or permitted ranges) for initiating and/or for completing an actuation session, a total time period over which device adjustment should be performed (e.g., 1 day, 1 week, 6 weeks, 1 month, 3 months), and/or other time related parameters),
b. an actuation session duration or a permitted range thereof,
c. a torque range or limit to be generated by an actuator; and/or other operational parameters of the actuator which affect, directly or indirectly, the axial adjustment of the strut, such as: current consumption of the actuator, voltage, rotation speed of the actuator, or other,
d. a desired change in the length of a strut or a permitted range thereof, for example, a length measured along the strut between the end attachments of the strut to the two portions of the frame,
e. a travel range of a strut, for example, a total distance along which the strut is planned to be adjusted,
f. length of one or more of the adjustable struts,
g. actuator current value of one or more actuators,
h. torques applied by actuators onto one or more of the adjustable struts,
i. force needed to move one or more of the adjustable struts.

FIG. 3 is an example of an actuation plan for adjustment of a plurality of struts of the bone fixation device, according to embodiments of the presently disclosed subject matter.

In some embodiments, the actuation plan defines, for the plurality of actuators, one or more parameters based on which operation of the actuator is carried out. Such parameters include the operational parameters exemplified above, and for example:
a. the length by which the strut associated with the actuator should be lengthened or shortened.
b. the total length of the strut following adjustment.
c. the adjustment initiation time and/or designated completion time.

Similarly, a status, for example a status report, for the bone fixation device, may include one or more of the operational parameters exemplified in this description, additional sensor readings, parameters, ranges, current data, historical data, raw data, processed data, and the like. A current status (report) may include data indicative of the operation and status of a current actuation plan on the bone fixation device. For example, the current status may include data indicative of one or more operational parameters (e.g., an actual length of the adjustable struts) at the time of the status report. The current status may also include history data related to one or more operational parameter of the bone fixation device.

In the example shown, a single day actuation plan including six actuations is presented. The actuation plan defines for each of six struts and for each actuation the time of the adjustment, and the length of the strut following adjustment.

In an exemplary actuation plan, 1-20 actuation sessions may be carried out in a single day. The total length by which the strut is adjusted over a day is, for example, up to 1 mm. In each actuation session, the strut is adjusted by, for example, 0.05 mm to 1 mm. A duration of each actuation session ranges, for example, between 2-10 seconds, e.g., 3 seconds, 5 seconds, 8 seconds.

### First Embodiments

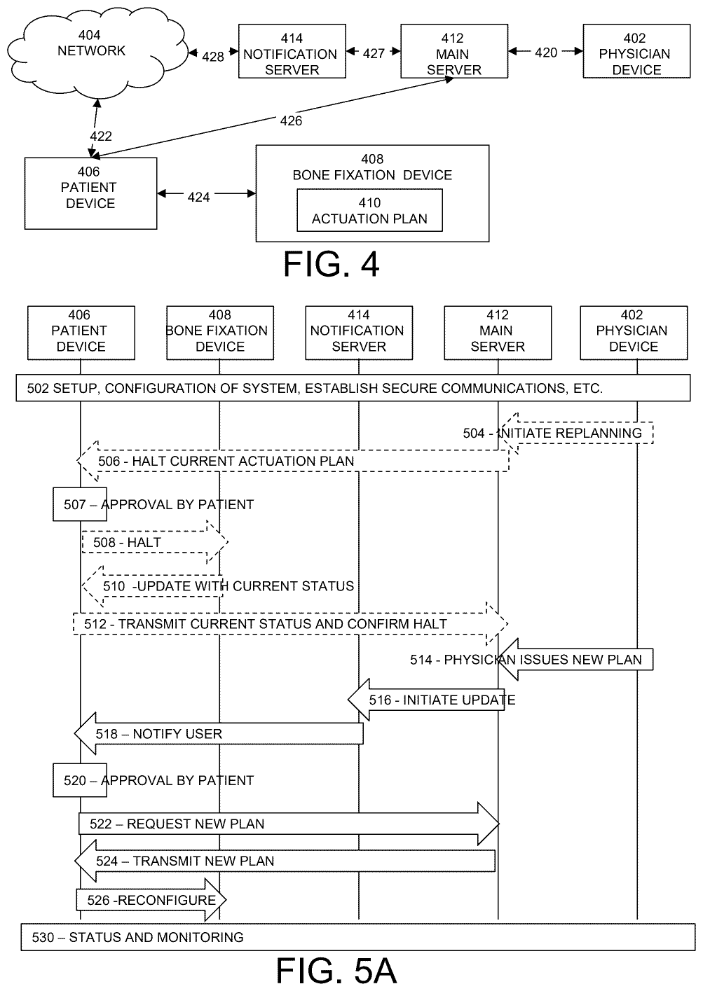

FIG. 4 illustrates an exemplary system configuration for remote reconfiguration of a bone fixation device of a patient. Features include authorization by a patient prior to reconfiguration, and secure operation over one or more communication channels with one or more servers. Reconfiguration may include halting, and resuming a current actuation plan, and/or loading/configuring and resuming with a new actuation plan. A physician device 402 is communicatively (operationally) coupled 420 to a main server 412 which is communicatively coupled 427 to a notification server 414. The main server 412 and notification server 414 may be deployed in, configured in, communicate via, and/or use a network 404. In the current non-limiting example, the notification server 414 is coupled 428 to/via the network 404 for communication 422 with a patient device 406. The main server 412 is coupled 426 to the patient device 406 optionally via the network 404. The network 404 is communicatively coupled 422 to the patient device 406 which is communicatively coupled 424 to the bone fixation device 408. The bone fixation device 408 may be configured with an actuation plan 410 (a single exemplary plan shown). The patient device 406 may be configured to interact with the bone fixation device 408 e.g., by configuring and reconfiguring the actuation plan 410 in the bone fixation device 408.

Devices such as the physician device 402, the main server 412, and the notification server 414 may be implemented on, in, and/or across one or more processing devices. For example, as individual computers or virtual cloud resources, each on an individual hardware processor, or two or more on a single hardware processor. In one embodiment, the main server 412 and the notification server 414 may be separate servers, that is, the system uses two servers. In another embodiment, the main server 412 and the notification server 414 may be implemented as modules on the same hardware processor. The main server 412 may be an AUTOSTRUT™ Server (available from DePuy Synthes of Johnson & Johnson, West Chester, Pennsylvania, USA). The patient device 406 may be a personal electronic device such as a mobile phone. The patient device 406 may include an application (for example an Android or Apple app) configured for implementing a portion of the described methods. The network 404 may be one or more interconnected networks, whether dedicated or distributed. Non-limiting examples include intranets, the Internet, cellular data communications networks, and switched telephonic networks or systems. Connections to the network 404 may be continuous or may be intermittent, only providing for a connection when requested by a sending or receiving device. While the current exemplary configuration shows the main server 412 communicating via the patient device 406 to the bone fixation device 408, the main server 412 may also be communicatively coupled to the bone fixation device 408. In one embodiment a single communication channel may be used by the patient device 406 to communicate with both the main server 412 and the notification server 414. In another embodiment, multiple, such as two, communication channels may be used by the patient device 406, a first channel to communicate with the main server 412 and another (second) channel to communicate with the notification server 414.

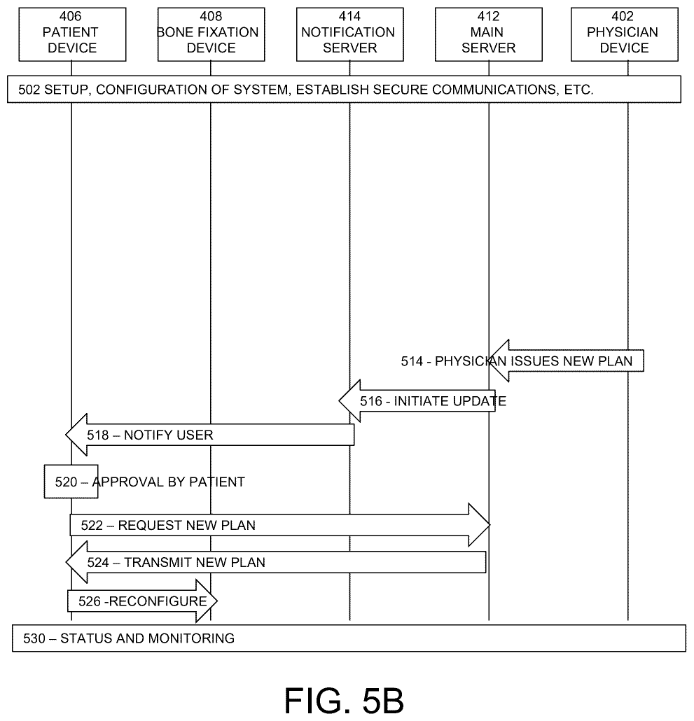

FIG. 5A illustrates a method and flow of communications with the exemplary system for remote reconfiguration of a bone fixation device of a patient. In general, the bone fixation device may include a frame connectible to bone tissue, a plurality of adjustable struts connected to the frame, and a control system including: a plurality of actuators associated with the plurality of adjustable struts, each actuator configured for adjusting a length of at least one adjustable strut.

In general, a method for remote reconfiguration of a bone fixation device 408 of a patient may include generating, on the main server 412, a new actuation plan for the bone fixation device 408, the new actuation plan including operational parameters of the bone fixation device, notifying the patient device 406 that the new actuation plan is available on the main server 412, requesting from a patient (not shown in the figures) on the patient device 406 an approval of the new actuation plan. Upon receiving the patient's approval, requesting by the patient device 406, from the main server 412, the new actuation plan from the main server 412, and reconfiguring the bone fixation device 408 with the new actuation plan 410.

In more detail, the bone fixation device frame may include two frame portions formed as two rings and the plurality of adjustable struts includes six struts which interconnect the two rings. The bone fixation device 408 may include processing circuitry. The processing circuitry may include at least one processor. The bone fixation device processing circuitry may be configured to receive, store, and execute the new actuation plan to signal the plurality of actuators to adjust the struts according to the new actuation plan. The bone fixation device circuitry may be configured to generate the current status of the bone fixation device 408. The bone fixation device circuitry may further be configured to communicate the current status to the patient device 406.

In step 502, setup may be done as appropriate for enabling the devices involved to communicate and function properly together. For example, registration, authentication, and authorization of the devices with each other may be performed. For example, setting up the physician device 402 with the main server 412, the main sever 412 with the notification server, the notification server 414 with the patient device 406, the main server 412 with the patient device 406, and the patient device 406 with the bone fixation device 408.

Optionally, the method may include step 504 to initiate replanning. The physician device 402 initiates replanning by transmitting to the main server 412. Transmitting may include a token, message, signal, etc., as appropriate for the specific implementation. For example, a physician in consultation with a patient, new factors in the treatment, review of the treatment progress such as review of one or more status reports from the bone fixation device, may indicate or require a change in the treatment, and therefore a change in the actuation plan.

Optionally, the method may include step 506 halt the current actuation plan, that is, halting operation of the bone fixation device 408. The main server 412 initiates halting the current actuation plan by transmitting a halt command to the patient device 406. Optionally, halting may include notifying the user that a halt has been requested (e.g., through the notification server) and/or requesting user approval for the halt.

In step 507 (similar to step 520) approval by patient, an approval of the halt is requested from the patient on the patient device 406.

In step 508 halt, upon receiving the patient's approval, the patient device 406 transmits a halt command to the bone fixation device 408. Halting the current actuation plan may alternatively/additionally be in response to receiving a current status (based on analysis of the current status), prior to, or as part of initiating an update, for example prior to reconfiguring the bone fixation device, temporarily halting the current actuation plan 410 of the bone fixation device 408.

In a case (step 507) where the patient does not approve the halt, the method and system may be configured to implement one or more options such as notifying the physician of the patient's lack of approval, sending a current status to the physician, etc.

Optionally, in step 510 update with current status, the bone fixation device 408 transmits a current status (status report) to the patient device 406. In a case where a halt command has been initiated, the status may include (in combination or in parallel with operational parameters) confirmation that the bone fixation device 408 has been halted. The current status may be generated and transmitted in a variety of ways, depending on the specifics of system requirements and implementation. For example, the current status may be pushed from the bone fixation device 408 or pulled by the patient device 406, the main server 412, or the physician device 402, periodically, aperiodically, on-demand, and/or automatically.

Optionally, in step 512 the current status (optionally with confirmation of halt) is transmitted from the patient device 406 to the main server 412. Prior to generating the new actuation plan, the main server 412 may receive a current status of the bone fixation device 408 from the patient device 406, the current status including data indicative of the operation and status of a current plan 410 on the bone fixation device 408.

In step 514 the physician issues a new actuation plan. The issuing from the physician device 402 is to the main server 412. Issuing 514 may include a new actuation plan or a treatment plan to be processed by the main server 412 to generate the new actuation plan. The bone fixation device 408 may be configured with a current actuation plan 410 and generating the new actuation plan may be based on the current actuation plan. Generating the new actuation plan may be based on the current status.

If a halt has been implemented and the halt duration is shorter than a specified amount of time (e.g., 12 or 24 hours), in some cases, the new actuation plan may be identical to the current one. The device may be configured to catch up to the current actuation plan by implementing adjustments at an accelerated pace. This allows the treatment to remain on track without significant delays, even after a temporary halt.

In step 516 initiate update, the main server 412 initiates an update of the actuation plan, including transmitting to the notification server 414. Depending on specifics of implementation, the transmitting may include a token, message, signal, etc., patient identifiers, actuation plan identifiers, authentication keys (tokens), authorization keys (tokens), verification keys (tokens), and in some embodiments the new actuation plan. Initiating an update 516 may include subsequent to generating the new actuation plan, initiating, by the main server 412, notifying the patient device 406. Alternatively, the main server 412 may notify (directly) the patient device 406.

In step 518 notify user, the notification server 414 transmits a notification to the patient device 406 that a new actuation plan is available. The notification may be pushed from the notification server 414 or pulled by the patient device 406. For example, in cases where communication is not constant or interrupted (network goes down, offline, patient device 406 is powered off, etc.) when communication is restored, the devices may push/pull to transmit/receive updates.

In step 520 approval by patient, an approval of the new actuation plan is requested from the patient on the patient device 406. The approval may be implemented as an indicator, token, key, signal, etc.

In step 522 request new plan, upon receiving the patient's approval, requesting by the patient device 406, from the main server 412, the new actuation plan from the main server 412.

In a case where the patient does not approve the new actuation plan, the method and system may be configured to implement one or more options such as: Halting the operation of the current actuation plan and/or notifying the physician of the patient's lack of approval.

In step 524 transmit new plan, the main server 412 transmits the new actuation plan to the patient device 406.

In step 526 reconfigure, the new actuation plan is transmitted from the patient device 406 to the bone fixation device 408 and the bone fixation device 408 is reconfigured based on the new actuation plan 410.

In step 530 status and monitoring, depending on specific system implementation, a variety of monitoring and status updates may be executed. This step may include sending confirmation from the bone fixation device 408 that the new actuation plan is in operation to the physician device 402 (via the patient device 406 and the main server 412). Status and monitoring may include the bone fixation device 408, the patient device 406, associated sensors (time, temperature, movement), and patient feedback. For example, the bone fixation device 408 may periodically generate a current status report which is pushed via the patient device 406 to the main server 412 and/or physician device 402 for automatic review, processing, and/or physician review. Based on the monitoring and status, as described above, replanning may be initiated based on the status of operation of the current actuation plan.

In an alternative embodiment, a first secure communication channel (428, 422) is configured (setup) between the notification server 414 and the patient device 406. Communications such as notifying the user 518 may be via the first secure communication channel (428, 422).

In an alternative embodiment, a second secure communication channel (426) is configured (setup) between the main server 412 and the patient device 406. Communications such as requesting, transmitting, and receiving the new actuation plan may be via the second secure communication channel (426).

In an alternative embodiment, the main server 412 may communicate the new actuation plan via a primary communication channel with the patient device 406, and information other than the new actuation plan is communicated via a secondary communication channel. For example, a user authentication key may be transmitted from the main server 412, the notification server 414, the physician device 402, or the physician via the secondary communication channel. Non limiting example of the secondary communication channel include channels that are parallel, out-of-band from communication channel 426, SMS, application message, voice phone call, etc. In an alternative embodiment, the primary communication channel may be the second secure communication channel, and the secondary communication channel may be the first secure communication channel. In an alternative embodiment, the primary communication channel may be the first secure communication channel, and the secondary communication channel may be the second secure communication channel. Reconfiguring of the bone fixation device 408 may include authentication of the new actuation plan using the user authenticating key.

In an alternative embodiment, notifying 518 the patient device includes sending an authenticating key to the patient device 406 and requesting the new actuation plan 522 includes sending the authenticating key from the patient device 406 to the main server 412.

In an alternative embodiment, notifying the patient device 406 includes sending an identifier to the patient device 406, and requesting the new actuation plan includes sending the identifier from the patient device 406 to the main server 412.

The system for remote reconfiguration of a bone fixation device 408 of a patient may include a patient device 406 including at least one processing circuitry for remote reconfiguration of the bone fixation device, the at least one processing circuitry configured for receiving, from the main server 412, a notification that a new actuation plan is available on the main server 412. The at least one processing circuitry is further configured for requesting from a patient an approval of the new actuation plan, and upon receiving the patient's approval, requesting, from the main server 412, the new actuation plan from the main server 412, then receiving, from the main server 412, the new actuation plan, and reconfiguring the bone fixation device 408 with the new actuation plan.

The at least one processing circuitry may be further configured for sending the current status of the bone fixation device to the main server 412.

The at least one processing circuitry may be further configured to receive the notification via a push notification.

The system may include the notification server 414 communicatively coupled to the patient device 406, and the at least one processing circuitry is further configured to receive the notification from the notification server 414.

The at least one processing circuitry may be further configured to receive the notification via the first secure communication channel between the notification server 414 and the patient device 406, request the new actuation plan via the second secure communication channel, and receive the new actuation plan via the second secure communication channel from the main server 412.

The at least one processing circuitry may be further configured for, prior to reconfiguring the bone fixation device 408, receiving from the main server 412 instructions to temporarily halt a current actuation plan of the bone fixation device.

The at least one processing circuitry may be further configured for receiving, from the main server 412, via the primary communication channel, the new actuation plan; receiving a user authenticating key via the secondary communication channel, and reconfiguring the bone fixation device 408 based on authentication of the new actuation plan using the user authenticating key.

The at least one processing circuitry may be further configured for receiving, as part of the notification, an authenticating key, and requesting the new actuation plan includes sending the authenticating key from the patient device 406 to the main server 412.

The at least one processing circuitry may be further configured for receiving, as part of the notification, an identifier, and requesting the new actuation plan includes sending the identifier from the patient device to the main server 412.

The at least one processing circuitry may be further configured for receiving, transmitting, and requesting with the main server 412.

FIG. 5B illustrates a method and flow of communications with the exemplary system for remote reconfiguration of a bone fixation device of a patient. The method and flow of the current figure is similar as described with reference to FIG. 5A, with same element numbers having similar descriptions. After setup 502, the physician issues a new plan 514, which initiates an update 516, and notifies the user 518. Upon receiving patient approval 520, the new plan is requested 522 and transmitted 524 for configuration 526 on the bone fixation device 408. As described elsewhere in this description, the actuation plan may include any information necessary to implement a treatment regimen with the bone fixation device 408, including for example, parameters for the control system, actuator parameters, and general parameters such as timing. Thus, the method of the current figure may be repeated to implement pausing operation of the bone fixation device, resuming operation with the current actuation plan or halting operation, reconfiguring with a new actuation plan, resuming operation with the new actuation plan, and other functions.

### Other Embodiments

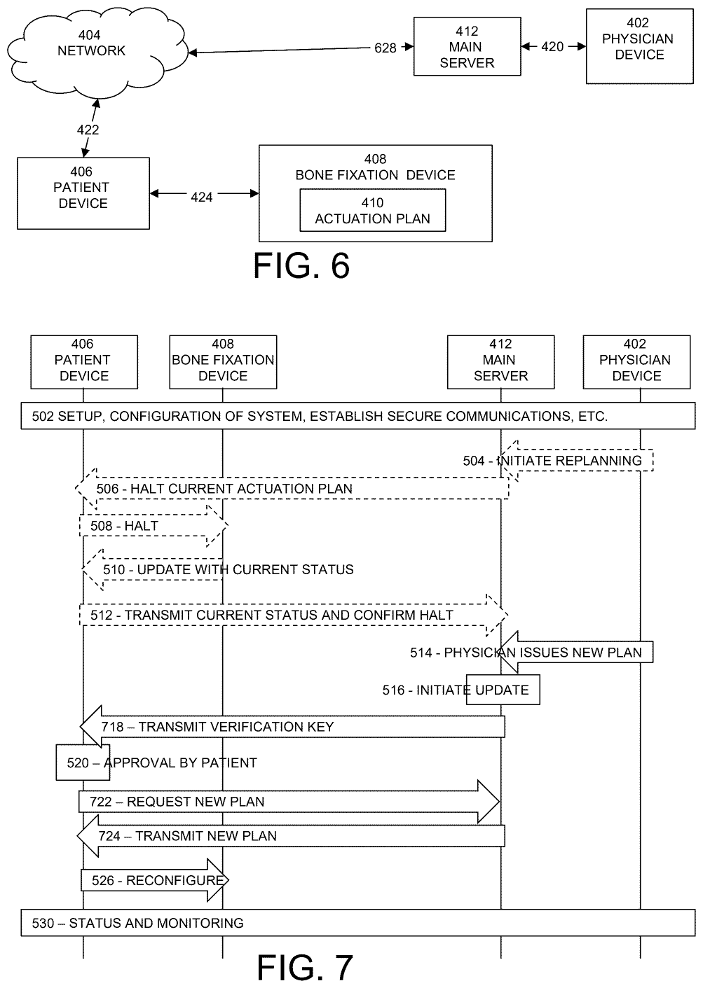

FIG. 6 illustrates an exemplary system configuration for remote reconfiguration of a bone fixation device of a patient. Features include authorization by a patient prior to reconfiguration, and secure operation over one or more communication channels with one or more servers. Authorization may be via techniques such as two-factor authentication or a one-time password. The configuration of elements is similar to the above description in reference to FIG. 4, with the main server 412 coupled 628 via the network 404 to the patient device 406. The current embodiment may use one or more communication channels communicatively coupled to the main server 412 which is implemented on one or more hardware processors.

FIG. 7 illustrates a method and flow of communications with the exemplary system for remote reconfiguration of a bone fixation device of a patient. The method and flow of the current figure is similar as described with reference to FIG. 5A, with same element numbers having similar descriptions.

In general, a method for remote reconfiguration of a bone fixation device 408 of a patient may include generating, on the main server 412, a new actuation plan for the bone fixation device 408, notifying the patient device 406 that the new actuation plan is available, and requesting from a patient on the patient device 406 an approval of the new actuation plan. Upon receiving the patient's approval, reconfiguring the bone fixation device 408 with the new actuation plan.

In more detail, in step 718 transmit verification key, a verification key may be transmitted from the main server 412 to the patient device 406, prior to step 520 requesting approval by the patient. In an alternative embodiment, prior to notifying the patient device 406 that the new actuation plan is available, transmitting, from the main server 412 to a patient device 406, the new actuation plan. Step 718 may also include notifying the patient (sending a notification to the patient device 406) from the main server 412, that the new actuation plan is available.

In step 722 request new plan, upon receiving the patient's approval, requesting by the patient device 406, from the main server 412, using the verification key, the new actuation plan. Thus, in one embodiment receiving the verification key on the patient device 406 and using the verification key to request the new actuation plan from the main server 412 implements two-factor authentication. The verification key may be a password/passcode and may be supplied by the physician.

In step 724 transmit new plan, similar to step 524, a new actuation plan is transmitted from the main server 412 to the patient device 406.

In an alternative embodiment, notifying the patient device 406 that the new actuation plan is available is from a notification server 414.

In an alternative embodiment, requesting by the patient device 406, from the main server 412, the new actuation plan may include use of a password in the request. Thus, in one embodiment implementing password authentication. The new actuation plan may be requested and/or transmitted via the primary communication channel and the password may be set and/or transmitted via the secondary communication channel (not shown in the current figure).

The system for remote reconfiguration of a bone fixation device of a patient may include the patient device 406 including at least one processing circuitry for remote reconfiguration of the bone fixation device 408, the at least one processing circuitry configured for receiving, from the communicatively coupled main server 412, a notification that a new actuation plan is available on the main server 412, and requesting from a patient an approval of the new actuation plan, and upon receiving the patient's approval, reconfiguring the bone fixation device 408 with the new actuation plan.

The at least one processing circuitry may be further configured for receiving from the main server 412 the new actuation plan.

The at least one processing circuitry may be further configured for receiving from the main server 412 a verification key.

The at least one processing circuitry may be further configured for, upon receiving the patient's approval, requesting from the main server 412, using the verification key, the new actuation.

The at least one processing circuitry may be further configured for upon receiving the patient's approval, requesting by the patient device 406, from the main server 412, the new actuation plan from the main server 412.

The system may further include a notification server 414 (not shown in the current figure), wherein the least one processing circuitry is further configured for receiving from the notification server 414, the notification that the new actuation plan is available.

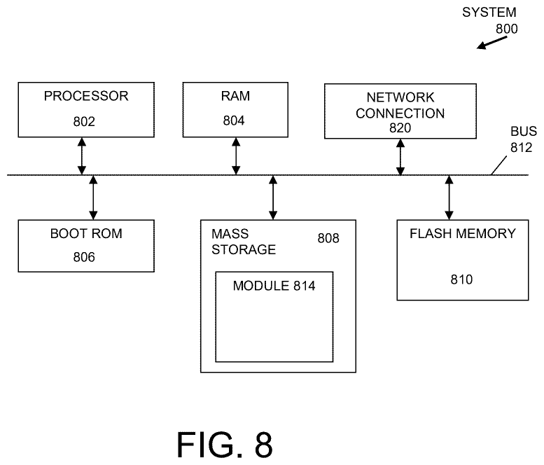

FIG. 8 is a high-level partial block diagram of an exemplary system 800 configured to implement the servers and/or devices of the present disclosure. System (processing system) 800 includes a processor 802 (one or more) and four exemplary memory devices: a RAM 804, a boot ROM 806, a mass storage device (hard disk) 808, and a flash memory 810, all communicating via a common bus 812. As is known in the art, processing and memory can include any computer readable medium storing software and/or firmware and/or any hardware element(s) including but not limited to field programmable logic array (FPLA) element(s), hard-wired logic element(s), field programmable gate array (FPGA) element(s), and application-specific integrated circuit (ASIC) element(s). Any instruction set architecture may be used in processor 802 including but not limited to reduced instruction set computer (RISC) architecture and/or complex instruction set computer (CISC) architecture. A module (processing module) 814 is shown on mass storage 808, but as will be obvious to one skilled in the art, could be located on any of the memory devices.

Mass storage device 808 is a non-limiting example of a non-transitory computer-readable storage medium bearing computer-readable code for implementing the remote reconfiguration of a bone fixation device methodology described herein. Other examples of such computer-readable storage media include read-only memories such as CDs bearing such code.

System 800 may have an operating system stored on the memory devices, the ROM may include boot code for the system, and the processor may be configured for executing the boot code to load the operating system to RAM 804, executing the operating system to copy computer-readable code to RAM 804 and execute the code.

Network connection 820 provides communications to and from system 800. Typically, a single network connection provides one or more links, including virtual connections, to other devices on local and/or remote networks. Alternatively, system 800 can include more than one network connection (not shown), each network connection providing one or more links to other devices and/or networks.

System 800 can be implemented as a server or client respectively connected through a network to a client or server.

### General

As used within this document, the term “about” may refer to ±20% The terms “comprises”, “comprising”, “includes”, “including”, “having” and their conjugates may refer to “including but not limited to”.

The term “consisting of”may refer to “including and limited to”.

As used herein, singular forms, for example, “a”, “an” and “the” include plural references unless the context clearly dictates otherwise. The term “each” may not be exclusively understood as referring to each and every, and when technically relevant may also refer to “at least some”.

Within this application, various quantifications and/or expressions may include use of ranges. Range format should not be construed as an inflexible limitation on the scope of the present disclosure. Accordingly, descriptions including ranges should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within the stated range and/or subrange, for example, 1, 2, 3, 4, 5, and 6. Whenever a numerical range is indicated within this document, it is meant to include any cited numeral (fractional or integral) within the indicated range.

It is appreciated that certain features which are (e.g., for clarity) described in the context of separate embodiments, may also be provided in combination in a single embodiment. Where various features of the present disclosure, which are (e.g., for brevity) described in a context of a single embodiment, may also be provided separately or in any suitable sub-combination or may be suitable for use with any other described embodiment. Features described in the context of various embodiments are not to be considered essential features of those embodiments, unless the embodiment is inoperative without those elements.

Although the present disclosure has been described in conjunction with specific embodiments thereof, it is evident that many alternatives, modifications, and variations will be apparent to those skilled in the art. Accordingly, this application intends to embrace all such alternatives, modifications and variations that fall within the spirit and broad scope of the appended claims.

Where section headings are used in this document, they should not be interpreted as necessarily limiting.

The following is a non-exclusive list of some exemplary examples of the disclosure. The present disclosure also includes examples which include fewer than all the features in an example and examples using features from multiple examples, even if not listed below.

In an example, a method for remote reconfiguration of a bone fixation device of a patient, the bone fixation device including a frame connectible to bone tissue, a plurality of adjustable struts connected to the frame, and a control system including: a plurality of actuators associated with the plurality of adjustable struts, each actuator configured for adjusting a length of at least one adjustable strut, the method including:
(a) generating, on a main server, a new actuation plan for the bone fixation device, the new actuation plan including operational parameters of the bone fixation device,
(b) notifying a patient device that the new actuation plan is available on the main server,
(c) requesting from a patient on the patient device an approval of the new actuation plan,
(d) upon receiving the patient's approval, requesting by the patient device the new actuation plan from the main server, and
(e) reconfiguring the bone fixation device with the new actuation plan.

In another example, the bone fixation device is configured with a current actuation plan, and generating the new actuation plan is based on the current actuation plan.

In another example, the new actuation plan sets one or more of the following operational parameters or indicators thereof:
(a) length of one or more of the adjustable struts,
(b) actuator current value of one or more actuators,
(c) torques applied by actuators onto one or more of the adjustable struts,
(d) timing of one or more adjustments,
(e) durations of one or more adjustments.

In another example, prior to generating the new actuation plan, receiving on the main server a current status of the bone fixation device from the patient device, the current status including data indicative of the operation and status of a current plan on the bone fixation device.

In another example, the current status of the bone fixation device includes one or more of the following operational parameters or indicators thereof:
(a) length of one or more of the adjustable struts,
(b) actuator current value of one or more actuators in one or more prior adjustments,
(c) torques applied by actuators onto one or more of the adjustable struts in one or more prior adjustments,
(d) force needed to move one or more of the adjustable struts in one or more prior adjustments,
(e) timing of one or more prior adjustments,
(f) durations of one or more prior adjustments,
(g) start time of one or more prior adjustments, and
(h) end time of one or more prior adjustments.

In another example, generating the new actuation plan is based on the current status.

In another example, the bone fixation device includes circuitry configured to generate the current status.

In another example, subsequent to generating the new actuation plan, initiating, by the main server, notifying the patient device.

In another example, the notifying the patient device uses a push notification.

In another example, the patient device is registered with one or more of:
(a) the main server, and
(b) a notification server.

In another example, further including a notification server, and notifying the patient device is via the notification server.

In another example, further including a first secure communication channel between the notification server and the patient device, and the notifying the patient device is via the first secure communication channel.

In another example, further including a second secure communication channel between the main server and the patient device, and the requesting the new actuation plan is via the second secure communication channel.

In another example, the new actuation plan is transmitted via the second secure communication channel from the main server to the patient device.

In another example, prior to reconfiguring the bone fixation device, transmitting from the main server to the patient device, instructions to temporarily halt a current actuation plan of the bone fixation device.

In another example, including:
(a) transmitting, by the main server, via a primary communication channel, to the patient device, the new actuation plan; and
(b) transmitting a user authenticating key to the patient device via a secondary communication channel,
and wherein reconfiguring the bone fixation device includes authentication of the new actuation plan using the user authenticating key.

In another example:
(i) notifying the patient device includes sending an authenticating key to the patient device, and
(ii) requesting the new actuation plan includes sending the authenticating key from the patient device to the main server.

In another example:
(i) notifying the patient device includes sending an identifier to the patient device, and
(ii) requesting the new actuation plan includes sending the identifier from the patient device to the main server.

In another example, the bone fixation device includes circuitry configured to receive, store, and execute the new actuation plan to signal the plurality of actuators to adjust the struts according to the new actuation plan.

In another example, the patient device is a mobile device.

In another example, further including, prior to generating the new actuation plan:
(a) generating, on a main server, a halt command for the bone fixation device, the halt command including operational parameters for ceasing operations of the bone fixation device,
(b) notifying the patient device that the halt command has been generated,
(c) requesting from the patient on the patient device an approval of the halt command, and
(d) upon receiving the patient's approval, transmitting the halt command to the bone fixation device.

In another example, the new actuation plan includes one or more operational parameters for implementing a function selected from the group consisting of:
(a) halting operation of one or more portions of the bone fixation device, and
(b) resuming operation of one or more portions of the bone fixation device.

In an example, a system for remote reconfiguration of a bone fixation device of a patient, the bone fixation device including a frame connectible to bone tissue, a plurality of adjustable struts connected to the frame, and a control system including: a plurality of actuators associated with the plurality of adjustable struts, each actuator configured for adjusting a length of at least one adjustable strut, the system including:

a patient device including at least one processing circuitry for remote reconfiguration of the bone fixation device, the at least one processing circuitry configured for:
(a) receiving, from a communicatively coupled main server, a notification that a new actuation plan is available on the main server, the new actuation plan including operational parameters of the bone fixation device,
(b) requesting from a patient an approval of the new actuation plan,
(c) upon receiving the patient's approval, requesting, from the main server, the new actuation plan from the main server,
(d) receiving, from the main server, the new actuation plan, and
(e) reconfiguring the bone fixation device with the new actuation plan.

In another example, the at least one processing circuitry is further configured for:

generating a current status of the bone fixation device, the current status including data indicative of the operation and status of a current plan on the bone fixation device.

In another example, the at least one processing circuitry is further configured for sending the current status of the bone fixation device to the main server.

In another example, the at least one processing circuitry is further configured to receive the notification via a push notification.

In another example, the patient device is registered with one or more of:
(a) the main server, and
(b) a communicatively coupled notification server.

In another example, further including a notification server communicatively coupled to the patient device, and the at least one processing circuitry is further configured to receive the notification from the notification server.

In another example, further including a first secure communication channel between the notification server and the patient device, and the notification is received by the at least one processing circuitry via the first secure communication channel.

In another example, further including a second secure communication channel between the main server and the patient device, and the at least one processing circuitry is further configured to request the new actuation plan via the second secure communication channel.

In another example, the at least one processing circuitry is further configured to receive the new actuation plan is via the second secure communication channel from the main server.

In another example, the at least one processing circuitry is further configured for: prior to reconfiguring the bone fixation device, receiving from the main server instructions to temporarily halt a current actuation plan of the bone fixation device.

In another example, the at least one processing circuitry is further configured for:
(a) receiving, from the main server, via a primary communication channel, the new actuation plan; and
(b) receiving a user authenticating key via a secondary communication channel, and
(c) reconfiguring the bone fixation device based on authentication of the new actuation plan using the user authenticating key.

In another example, the at least one processing circuitry is further configured for:
(a) receiving, as part of the notification, an authenticating key, and
(b) requesting the new actuation plan includes sending the authenticating key from the patient device to the main server.

In another example, the at least one processing circuitry is further configured for:
(a) receiving, as part of the notification, an identifier, and
(b) requesting the new actuation plan includes sending the identifier from the patient device to the main server.

In another example, further including the main server communicatively coupled to the patient device, and the at least one processing circuitry is further configured for receiving, transmitting, and requesting with the main server.

In another example, the bone fixation device includes circuitry configured to receive, store, and execute the new actuation plan to signal the plurality of actuators to adjust the struts according to the new actuation plan.

In another example, the bone fixation device includes circuitry configured to generate the current status.

In another example, the patient device is a mobile device.

In an example, a method for remote reconfiguration of a bone fixation device of a patient, the bone fixation device including a frame connectible to bone tissue, a plurality of adjustable struts connected to the frame, and a control system including: a plurality of actuators associated with the plurality of adjustable struts, each actuator configured for adjusting a length of at least one adjustable strut, the method including:
(a) generating, on a main server, a new actuation plan for the bone fixation device, the new actuation plan including operational parameters of the bone fixation device,
(b) notifying a patient device that the new actuation plan is available,
(c) requesting from a patient on the patient device an approval of the new actuation plan,
(d) upon receiving the patient's approval, reconfiguring the bone fixation device with the new actuation plan.

In another example, prior to notifying the patient device that the new actuation plan is available, transmitting, from the main server to a patient device, the new actuation plan.

In another example, further including transmitting a verification key from the main server to the patient device.

In another example, upon receiving the patient's approval, requesting by the patient device, from the main server, using the verification key, the new actuation plan.

In another example, upon receiving the patient's approval, requesting by the patient device, from the main server, the new actuation plan from the main server.

In another example, notifying the patient device that the new actuation plan is available is from a notification server.

In an example, a system for remote reconfiguration of a bone fixation device of a patient, the bone fixation device including a frame connectible to bone tissue, a plurality of adjustable struts connected to the frame, and a control system including: a plurality of actuators associated with the plurality of adjustable struts, each actuator configured for adjusting a length of at least one adjustable strut, the system including:

a patient device including at least one processing circuitry for remote reconfiguration of the bone fixation device, the at least one processing circuitry configured for:
(a) receiving, from a communicatively coupled main server, a notification that a new actuation plan is available on the main server, the new actuation plan including operational parameters of the bone fixation device,
(b) requesting from a patient an approval of the new actuation plan, and
(c) upon receiving the patient's approval reconfiguring the bone fixation device with the new actuation plan.

In another example, the least one processing circuitry is further configured for receiving from the main server the new actuation plan.

In another example, the least one processing circuitry is further configured for receiving from the main server a verification key.

In another example, the least one processing circuitry is further configured, upon receiving the patient's approval, requesting from the main server, using the verification key, the new actuation.

In another example, the least one processing circuitry is further configured for, upon receiving the patient's approval, requesting by the patient device, from the main server, the new actuation plan from the main server.

In another example, further including a notification server, and wherein the least one processing circuitry is further configured for receiving from the notification server, the notification that the new actuation plan is available.

In an example, a method for remote reconfiguration of a bone fixation device of a patient, the bone fixation device including a frame connectible to bone tissue, a plurality of adjustable struts connected to the frame, and a control system including: a plurality of actuators associated with the plurality of adjustable struts, each actuator configured for adjusting a length of at least one adjustable strut, the method including:
(a) generating, on a main server, a halt command for the bone fixation device, the halt command including operational parameters for ceasing operations of the bone fixation device,
(b) notifying a patient device that the halt command has been generated,
(c) requesting from a patient on the patient device an approval of the halt command,
(d) upon receiving the patient's approval, transmitting the halt command to the bone fixation device,
(e) generating, on the main server, a new actuation plan for the bone fixation device, the new actuation plan including operational parameters of the bone fixation device,
(f) notifying the patient device that the new actuation plan is available on the main server,
(g) requesting from the patient on the patient device an approval of the new actuation plan,
(h) upon receiving the patient's approval, requesting by the patient device the new actuation plan from the main server, and
(i) reconfiguring the bone fixation device with the new actuation plan.

1. A method for remote reconfiguration of a bone fixation device of a patient, the bone fixation device including a frame connectible to bone tissue, a plurality of adjustable struts connected to the frame, and a control system including: a plurality of actuators associated with the plurality of adjustable struts, each actuator configured for adjusting a length of at least one adjustable strut, the method comprising:
(a) generating, on a main server, a new actuation plan for the bone fixation device, the new actuation plan including operational parameters of the bone fixation device,
(b) notifying a patient device that the new actuation plan is available on the main server,
(c) requesting from a patient on the patient device an approval of the new actuation plan,
(d) upon receiving the patient's approval, requesting by the patient device the new actuation plan from the main server, and
(e) reconfiguring the bone fixation device with the new actuation plan.

2. The method according to claim 1, wherein the bone fixation device is configured with a current actuation plan, and generating the new actuation plan is based on the current actuation plan.

3. The method according to claim 1, further comprising, prior to generating the new actuation plan, receiving on the main server a current status of the bone fixation device from the patient device, the current status including data indicative of the operation and status of a current plan on the bone fixation device.

4. The method according to claim 3, wherein generating the new actuation plan is based on the current status.

5. The method according to claim 1, further comprising, subsequent to generating the new actuation plan, initiating, by the main server, notifying the patient device.

6. The method according to claim 1, further including a notification server, and notifying the patient device is via the notification server.

7. The method according to claim 6, further including a first secure communication channel between the notification server and the patient device, and the notifying the patient device is via the first secure communication channel.

8. The method according to claim 6, further including a second secure communication channel between the main server and the patient device, the requesting the new actuation plan is via the second secure communication channel, and the new actuation plan is transmitted via the second secure communication channel from the main server to the patient device.

9. The method according to claim 1, further comprising:
(a) transmitting, by the main server, via a primary communication channel, to the patient device, the new actuation plan; and
(b) transmitting a user authenticating key to the patient device via a secondary communication channel, and wherein reconfiguring the bone fixation device includes authentication of the new actuation plan using the user authenticating key.

10. The method according to claim 1, wherein:
(i) notifying the patient device includes sending an authenticating key to the patient device, and
(ii) requesting the new actuation plan includes sending the authenticating key from the patient device to the main server.

11. The method according to claim 1, wherein:
(i) notifying the patient device includes sending an identifier to the patient device, and
(ii) requesting the new actuation plan includes sending the identifier from the patient device to the main server.

12. The method according to claim 1, further including, prior to generating the new actuation plan:
(a) generating, on the main server, a halt command for the bone fixation device, the halt command including operational parameters for ceasing operations of the bone fixation device,
(b) notifying the patient device that the halt command has been generated,
(c) requesting from the patient on the patient device an approval of the halt command, and
(d) upon receiving the patient's approval, transmitting the halt command to the bone fixation device.

13. The method according to claim 1, wherein the new actuation plan includes one or more operational parameters for implementing a function selected from the group consisting of:
(a) halting operation of one or more portions of the bone fixation device, and
(b) resuming operation of one or more portions of the bone fixation device.

14. A system for remote reconfiguration of a bone fixation device of a patient, the bone fixation device including a frame connectible to bone tissue, a plurality of adjustable struts connected to the frame, and a control system including: a plurality of actuators associated with the plurality of adjustable struts, each actuator configured for adjusting a length of at least one adjustable strut, the system comprising:
a patient device comprising at least one processing circuitry for remote reconfiguration of the bone fixation device, the at least one processing circuitry configured for:
(a) receiving, from a communicatively coupled main server, a notification that a new actuation plan is available on the main server, the new actuation plan including operational parameters of the bone fixation device,
(b) requesting from a patient an approval of the new actuation plan,
(c) upon receiving the patient's approval, requesting, from the main server, the new actuation plan from the main server,
(d) receiving, from the main server, the new actuation plan, and
(e) reconfiguring the bone fixation device with the new actuation plan.

15. The system according to claim 14, wherein the at least one processing circuitry is further configured for:
generating a current status of the bone fixation device, the current status including data indicative of the operation and status of a current plan on the bone fixation device.

16. The system according to claim 14, further including a notification server communicatively coupled to the patient device, and the at least one processing circuitry is further configured to receive the notification from the notification server.

17. The system according to claim 16, further including a first secure communication channel between the notification server and the patient device, and the notification is received by the at least one processing circuitry via the first secure communication channel.

18. The system according to claim 16, further including a second secure communication channel between the main server and the patient device, and the at least one processing circuitry is further configured to request the new actuation plan via the second secure communication channel and receive the new actuation plan is via the second secure communication channel from the main server.

19. The system according to claim 14, wherein the at least one processing circuitry is further configured for:
(a) receiving, from the main server, via a primary communication channel, the new actuation plan; and
(b) receiving a user authenticating key via a secondary communication channel, and
(c) reconfiguring the bone fixation device based on authentication of the new actuation plan using the user authenticating key.

20. A method for remote reconfiguration of a bone fixation device of a patient, the bone fixation device including a frame connectible to bone tissue, a plurality of adjustable struts connected to the frame, and a control system including: a plurality of actuators associated with the plurality of adjustable struts, each actuator configured for adjusting a length of at least one adjustable strut, the method comprising:
(a) generating, on a main server, a halt command for the bone fixation device, the halt command including operational parameters for ceasing operations of the bone fixation device,
(b) notifying a patient device that the halt command has been generated,
(c) requesting from a patient on the patient device an approval of the halt command,
(d) upon receiving the patient's approval, transmitting the halt command to the bone fixation device,
(e) generating, on the main server, a new actuation plan for the bone fixation device, the new actuation plan including operational parameters of the bone fixation device,
(f) notifying the patient device that the new actuation plan is available on the main server,
(g) requesting from the patient on the patient device an approval of the new actuation plan,
(h) upon receiving the patient's approval, requesting by the patient device the new actuation plan from the main server, and
(i) reconfiguring the bone fixation device with the new actuation plan.
